(19) United States
(12) Patent Application Publication
(10) Pub. No.: US 2024/0325505 A1
(43) Pub. Date: Oct. 3, 2024

(54) Pharmaceutical Composition for Improving and Treating Leukotrichia and/or Alopecia and Preparation Method Thereof

(71) Applicant: Beijing University of Chemical Technology, Beijing (CN)

(72) Inventors: Qipeng YUAN, Beijing (CN); Li CHENG, Beijing (CN); Pengtao LIU, Beijing (CN)

(73) Assignee: Beijing University of Chemical Technology, Beijing (CN)

(21) Appl. No.: 18/288,797

(22) PCT Filed: Sep. 8, 2022
(86) PCT No.: PCT/CN2022/117854

§ 371 Date: Oct. 27, 2023

(30) Foreign Application Priority Data
Oct. 15, 2021 (CN) 202111202516.0

(51) Int. Cl. A61K 38/47; A61K 31/7028; A61P 17/00; A61P 17/14

(52) U.S. Cl. CPC A61K 38/47; A61K 31/7028; A61P 17/00; A61P 17/14; C12Y 302/01147

(57) ABSTRACT

The present disclosure relates to a pharmaceutical composition for improving and treating leukotrichia and/or alopecia and a preparation method thereof. In the present disclosure, the pharmaceutical composition includes glucoraphanin and myrosinase as active ingredients, where the glucoraphanin has a content of 0.1% to 50% and the myrosinase has a total enzyme activity of 0.1 mU to 10 U based on a total weight of the pharmaceutical composition. The pharmaceutical composition shows safety, rapidity, effective improvement and treatment of leukotrichia and/or alopecia, and wide application range for subjects.

### TECHNICAL FIELD

The present disclosure relates to a pharmaceutical composition for improving and treating leukotrichia and/or alopecia, including glucoraphanin and myrosinase as active ingredients. The present disclosure further relates to a preparation method of the pharmaceutical composition.

### BACKGROUND

The leukotrichia can be divided into physiological leukotrichia and pathological leukotrichia. The physiological leukotrichia is the senile leukotrichia, and the pathological leukotrichia can be divided into nutritional metabolism-caused leukotrichia, chemical leukotrichia, and hereditary leukotrichia.

Alopecia is divided into normal physiological alopecia and pathological alopecia. The normal physiological alopecia can keep the hair entering a catagen phase and the hair newly entering a growth phase in a constant dynamic balance, such that the normal number of hair can be maintained. However, the pathological alopecia causes abnormal or excessive hair loss. There are complex causes of the pathological alopecia, and the pathological alopecia includes, for example, androgenetic alopecia, neuropathic alopecia, endocrine alopecia, nutritional alopecia, and congenital alopecia.

At present, control methods for the leukotrichia and alopecia include traditional Chinese medicine therapy and Western medicine therapy. In Patent Document 1, traditional Chinese medicine believes that the following factors are related to leukotrichia: one is the deficiency of essence and blood: the deficiency of kidney essence cannot metabolize and produce Yin blood, and the deficiency of Yin blood causes the hair to lose nourishment, thus turning white gray. The second is excessive blood heat: emotional excitement causes the kidney failing to nourish liver, such that the liver-fire is strong and the blood is dry, and the excessive blood heat leads to the loss of nourishment of the hair roots, so the hair turns white prematurely. The third is liver stagnation and spleen dampness: the stagnation of liver qi causes damage to the heart and spleen, the damages of spleen lead to negligence of transportation and transformation, such that there is no source of qi and blood generation, and the hair becomes white.

Patent Document 2 disclosed a capsule for treating juvenile leukotrichia, including traditional Chinese medicines Fructus Ligustri Lucidi, Herba Ecliptae, Mulberry Fruit, Radix Glycyrrhizae, black sesame, and gelatin, where the capsule is processed through a technological process of capsule products. Patent Document 3 disclosed a traditional Chinese medicine composition and a granule thereof for blackening hair, including Fructus Ligustri Lucidi, Herba Ecliptae, Radix Polygoni Multiflori Praeparata, Rhizoma Polygonati, and black soybean, where the granule is processed through a technological process of granular pharmaceutical products. None of these traditional Chinese medicine therapies has found any component that shows a significant effect in controlling leukotrichia, while side effects caused by multiple components cannot be ignored and are also a common shortcoming of the traditional Chinese medicine therapies.

Regarding Western medicine therapy, Patent Document 4 disclosed a composition and a method for controlling or slowing down hair discoloration. The composition includes a catalase, an anti-oxidant, and a cosmetic product carrier, and has a pH value controlled at 2 to 6. A substance containing the composition acts directly on mid-section to root of the hair to control or slow down the graying of hair. However, an influence of the composition described in this document on melanocytes and tyrosinase has not been thoroughly studied, there is also a poor effect on preventing black hair from graving, and this composition may have potential side effects.

Non-patent Document 1 has also introduced that glucoraphanin, as a glucosinolate, can be extracted from natural products such as traditional Chinese medicine Semen Raphani and radish seeds. However, there is currently no report on the biological activity of glucosinolates on the human body, and the biological activity of these substances is generally reflected in their degradation products. The glucosinolate is a sulfur-containing secondary metabolite of cruciferous plants. Currently, more than 120 kinds of glucosinolates have been found in plants, and are water-soluble, non-volatile, and thermally-stable ionic compounds. If plant cells are damaged, a glucosinolate hydrolase, myrosinase (EC3.2.3.137), can be released to hydrolyze the glucosinolates to generate bisulfate, glucose, and a series of glucoside ligands. These ligands undergo intramolecular rearrangement to form isothiocyanates, thiocyanates, nitriles, and a small amount of epithionitriles.

### DOCUMENTS IN THE PRIOR ART

Patent Document 1: CN105660933A
Patent Document 2: CN104415101A
Patent Document 3: CN104606485A
Patent Document 4: U.S. Pat. No. 9,265,717B1
Non-patent Document 1: Halkier B. A., Gershenzon J. Biology and biochemistry of glucosinolates [J]. Annu. Rev. Plant Biol., 2006, 57:303-333.

### SUMMARY

### Technical Problem to be Solved by the Present Disclosure

It can be seen from the background that the current treatment of leukotrichia and/or alopecia at home and abroad has various defects and shortcomings such as large side effects and unsatisfactory treatment effects. Therefore, it is of great significance to find a drug that can safely, quickly, and effectively improve and treat leukotrichia and/or alopecia.

### Technical Solutions for Resolving the Technical Problem

In the present disclosure, during the application research of glucoraphanin, it is unexpectedly found that the combination of glucoraphanin and myrosinase has hair growth and hair blackening effects. In particular, after a composition containing glucoraphanin and myrosinase as active ingredients is made into water-soluble granules, tablets, capsules and other dosage forms and administered to a subject, the composition can improve and treat leukotrichia and/or alopecia in a shorter time than the prior art.

The inventors have not found a mechanism of action of the glucoraphanin combined with the myrosinase in improving and treating the leukotrichia and/or alopecia, and will conduct research on this aspect in the future. According to current analysis, this mechanism may be related to an antioxidant function of the glucoraphanin and myrosinase. This function improves the immunity of patients with leukotrichia and alopecia, thereby inhibiting leukotrichia and/or alopecia caused by excessive or abnormal responses of the immune system.

The present disclosure mainly relates to the following aspects:

[1]. The present disclosure provides a pharmaceutical composition for improving and treating leukotrichia and/or alopecia, including glucoraphanin and myrosinase as active ingredients, where the glucoraphanin has a content of 0.1% to 50% and the myrosinase has a total enzyme activity of 0.1 mU to 10 U based on a total weight of the pharmaceutical composition.

[2]. According to the pharmaceutical composition in [1], the glucoraphanin has a content of preferably 0.5% to 10%, more preferably 1% to 5%.

[3]. According to the pharmaceutical composition in [1], the myrosinase has a total enzyme activity of preferably 0.3 mU to 5 U, more preferably 0.5 mU to 1 U.

[4]. According to the pharmaceutical composition in any one of [1] to [3], the pharmaceutical composition is subjected to oral administration at a dosage of 5 mg/person/day to 500 mg/person/day based on an amount of the glucoraphanin.

[5]. According to the pharmaceutical composition in [4], the pharmaceutical composition is subjected to oral administration at a dosage of preferably 5 mg/person/day to 100 mg/person/day, more preferably 10 mg/person/day to 60 mg/person/day based on the amount of the glucoraphanin.

[6]. According to the pharmaceutical composition in any one of [1] to [5], the glucoraphanin and the myrosinase each are derived from one or more selected from the group consisting of a natural product extract, a microbial extract, a biological synthesis product, and a chemical synthesis product.

[7]. According to the pharmaceutical composition in [6], the natural product extract is extracted from a natural product being one or more selected from the group consisting of Brassica oleracea var. gemmifera Zenker, Brassica oleracea var. capitata Linnaeus, Brassica oleracea var. botrytis Linnaeus, Brassica rapa var. glabra Regel, kale, Brassica oleracea var. acephala DC., broccoli sprouts, Brassica oleracea var. albiflora Kuntze, cauliflower, Brassica juncea var. napiformis Pailleux et Bois, mustard, Brassica rapa L., Raphanus sativus L., Eruca vesicaria subsp. sativa (Miller) Thellung, and Nasturtium officinale R. Br. ex W. T. Aiton.

[8]. According to the pharmaceutical composition in any one of [1] to [7], the pharmaceutical composition is in the form of a tablet, a capsule, a pulvis, a powder, a water-soluble granule, a granule, an ointment, a patch, an emulsion, a liniment, a paste, an injection, a spray, a cream, a lotion, an oil, a suspension, a gel, or a tonic.

[9]. According to the pharmaceutical composition in any one of [1] to [8], the pharmaceutical composition is administered to a subject by oral administration, injection, skin administration, or mucosal administration.

[10]. The present disclosure further provides a preparation method of a pharmaceutical composition for improving and treating leukotrichia and/or alopecia, including: encapsulating glucoraphanin and myrosinase as active ingredients into the pharmaceutical composition, where the glucoraphanin has a content of 0.1% to 50% and the myrosinase has a total enzyme activity of 0.1 mU to 10 U based on a total weight of the pharmaceutical composition.

[11]. According to the preparation method of a pharmaceutical composition in [10], the preparation method includes the following steps:
preparing a glucoraphanin extract;
preparing a myrosinase extract;
mixing the glucoraphanin extract, an additive, and water to allow spray-drying to prepare a dried glucoraphanin powder;
mixing the myrosinase extract, the additive, and water to allow spray-drying to prepare a dried myrosinase powder; and
mixing the dried glucoraphanin powder and the dried myrosinase powder to obtain the pharmaceutical composition.

[12]. According to the preparation method of a pharmaceutical composition in or [11], the pharmaceutical composition is subjected to granulation by a granulator and then sub-packaging or tableting.

[13]. The present disclosure further provides use of glucoraphanin and myrosinase in preparation of a pharmaceutical composition for improving and treating leukotrichia and/or alopecia.

### Beneficial Effects of the Present Disclosure:

In the present disclosure, the pharmaceutical composition containing glucoraphanin and myrosinase as active ingredients exhibits a surprising effect in improving and treating leukotrichia and/or alopecia. The pharmaceutical composition can significantly promote the growth, repair, and increase of hair, quickly reduce the degree of alopecia, prevent the progression of alopecia, and promote gray and white hair to turn black from the roots. In addition, the pharmaceutical composition can also safely, quickly, and effectively improve and treat leukotrichia and/or alopecia, and is adaptable to subjects of different ages and symptoms.

In addition, the pharmaceutical composition is applicable to congenital and acquired leukotrichia, physiological and pathological leukotrichia, and physiological and pathological alopecia.

### BRIEF DESCRIPTION OF THE DRAWINGS

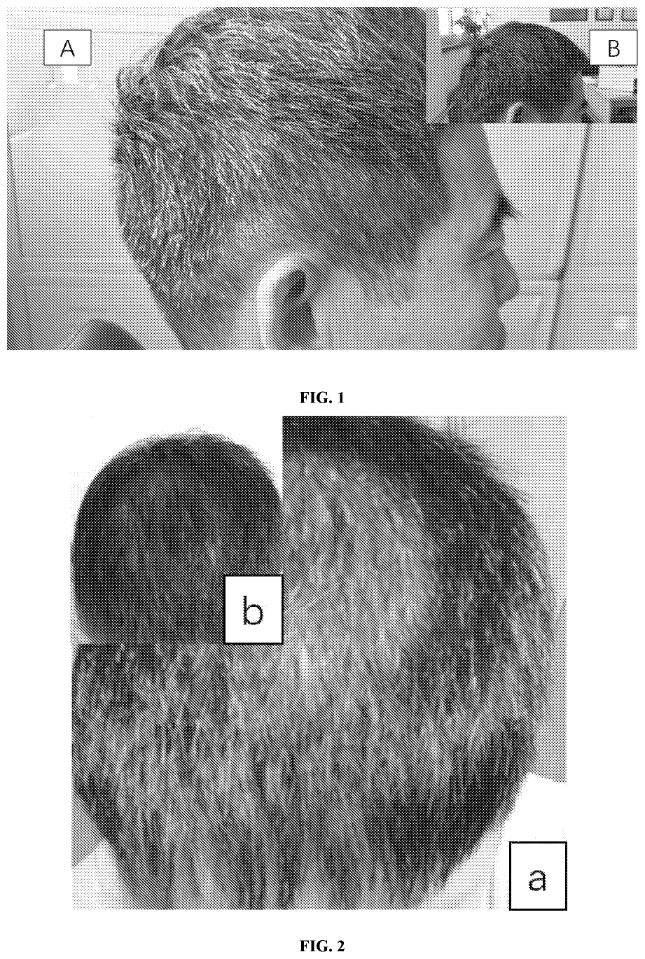

A and B in FIG. 1 show a comparison of hair photography of No. 3 subject in Table 1a with leukotrichia after taking the composition containing glucoraphanin and myrosinase for 1 month and before taking same, respectively; and

a and b in FIG. 2 show a comparison of hair photography of No. 4 subject in Table 1a with both leukotrichia and alopecia after taking the composition containing glucoraphanin and myrosinase for 1 month and before taking same, respectively.

### DETAILED DESCRIPTION OF THE EMBODIMENTS

In the present disclosure, the pharmaceutical composition includes glucoraphanin and myrosinase as active ingredients. There is no limitation on contents of the glucoraphanin and myrosinase, as long as an effect of improving and treating leukotrichia and/or alopecia can be achieved.

In the pharmaceutical composition of the present disclosure, the myrosinase mainly acts by promoting enzymatic hydrolysis of the substrate glucoraphanin. Therefore, the enzyme activity of the myrosinase and the content of the substrate glucoraphanin are particularly important for the composition to achieve the effect of improving and treating leukotrichia and/or alopecia.

The inventors have found that based on a total weight of the pharmaceutical composition: the glucoraphanin with a content of 0.1% to 50% and the myrosinase with a total enzyme activity of 0.1 mU to 10 U can effectively achieve the effect of improving and treating leukotrichia and/or alopecia.

The inventors have also found that when the content of glucoraphanin is less than 0.1%, the content of glucoraphanin is too low and a product obtained after the enzymatic reaction is insufficient to improve and treat leukotrichia and/or alopecia. When the content of glucoraphanin is higher than 50%, the effect of improving and treating leukotrichia and/or alopecia is not significantly improved. In addition, too much glucoraphanin makes subsequent spray-drying and other operations difficult, and is not conducive to the powder formation of the product. Moreover, too much glucoraphanin may cause the product to absorb moisture, which is not conducive to storage and affects the appearance and quality. Preferably, the glucoraphanin has a content of preferably 0.5% to 10%, more preferably 1% to 5%.

The enzyme activity of myrosinase can affect the enzymatic hydrolysis rate and conversion efficiency of the substrate glucoraphanin. The inventors have found that when a total enzyme activity of myrosinase is lower than 0.1 mU, glucoraphanin cannot be effectively enzymatically hydrolyzed. When the total enzyme activity is higher than 10 U, although the enzymatic hydrolysis is sufficient, a preparation cost of the composition becomes higher and a protein content also becomes higher. This makes subsequent spray-drying operations difficult and is not conducive to the powder formation of the product. Preferably, the myrosinase has a total enzyme activity of 0.3 mU to 5 U. Within this range, the myrosinase can efficiently decompose the glucoraphanin, and a resulting product has an appropriate viscosity, which is conducive to spray-drying into powder. More preferably, the myrosinase has a total enzyme activity of 0.5 mU to 1 U. Within this range, the myrosinase can maintain a high activity to effectively decompose the glucoraphanin and obtain a powder product with an excellent appearance.

Further researches have found that when the content of glucoraphanin is 0.5% to 10% and the total enzyme activity of myrosinase is 0.3 mU to 5 U, the combination of glucoraphanin and myrosinase can significantly improve and treat leukotrichia and/or alopecia in a relatively short period of time, such as within 2 to 3 months. More preferably, when the content of glucoraphanin is 1% to 5% and the total enzyme activity of myrosinase is 0.5 mU to 1 mU, a higher proportion of subjects can be improved and treated for their leukotrichia and/or alopecia in a short period of time.

In the present disclosure, the glucoraphanin and myrosinase can be prepared by any method known in the art, such as natural product extraction, microbial extraction, biological synthesis, or chemical synthesis, as long as the content of glucoraphanin and the total enzyme activity of myrosinase are within the above ranges, and the combination has the effect of improving and treating leukotrichia and/or alopecia.

In the present disclosure, a glucoraphanin extract and a myrosinase extract each are preferably extracted from a natural product, and the extracts can be obtained from seeds, flowers, stems, and leaves of cruciferous plants. More preferably, the natural product extract is extracted from a natural product being one or more selected from the group consisting of Brassica oleracea var. gemmifera Zenker, Brassica oleracea var. capitata Linnaeus, Brassica oleracea var. botrytis Linnaeus, Brassica rapa var. glabra Regel, kale, Brassica oleracea var. acephala DC., broccoli sprouts, Brassica oleracea var. albiflora Kuntze, cauliflower, Brassica juncea var. napiformis Pailleux et Bois, mustard, Brassica rapa L., Raphanus sativus L., Eruca vesicaria subsp. sativa (Miller) Thellung, and Nasturtium officinale R. Br. ex W. T. Aiton. Most preferably, the extracts are obtained from radish seeds.

In the present disclosure, a glucoraphanin compound is preferably used with the following structure:

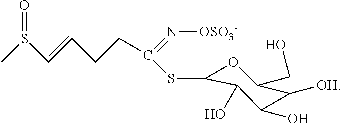

In the present disclosure, the pharmaceutical composition can preferably be prepared by the following method, but is not limited to the following method.

### <Preparation of the Glucoraphanin Extract>

### A. Crushing of Raw Materials

One or more of the seeds, flowers, stems, and leaves of cruciferous plants that serve as raw materials are crushed with a crusher, and resulting crushed materials are collected.

### B. Extraction of Glucoraphanin

The deionized water is heated to boiling, the crushed materials obtained in step A are added into the deionized water according to a solid-to-liquid ratio of 1:5 to 1:50, a resulting mixture is extracted with boiling water for 10 min to 30 min, and then filtered with a filter cloth to obtain a water extract. The extraction is repeated once, and an obtained glucoraphanin water extract is collected.

A dosage of activated carbon is determined according to a mass ratio of 10:1 to 10:5 between the activated carbon and the aqueous glucoraphanin extract, and the water extract is added into the activated carbon: a resulting mixture is stirred and adsorbed at 0° C. to 50° C. for 10 min to 60 min, and an activated carbon filter cake with the glucoraphanin adsorbed is obtained after filtration. The activated carbon filter cake is mixed with an aqueous solution of 1% to 30% methanol, ethanol, or acetonitrile at a solid-to-liquid ratio of 1:5 to 1:30; after an obtained mixture is adjusted with ammonia water and measured with a pH meter to have a pH value of 8 to 12, the mixture is eluted by stirring at 0° C. to 50° C. for 10 min to 60 min, and then filtered to obtain an eluate; and the eluate is distilled under reduced pressure and freeze-dried to obtain the glucoraphanin extract.

### <Preparation of the Myrosinase Extract>

The seeds, flowers, stems, and leaves of cruciferous plants not treated by high temperature are crushed, ultrasonically broken, and filtered with gauze to obtain a clear liquid. The myrosinase in the clear liquid is precipitated using a (NH₄)₂SO₄ aqueous solution with a saturation of 40% to 80%. A resulting myrosinase mixture containing precipitate is centrifuged at 5,000 r/min to 15,000 r/min for 15 min to 30 min, and a supernatant is discarded: an obtained precipitate is dialyzed overnight using a dialysis membrane with a molecular weight of 8,000 KD to 14,000 KD to obtain an enzyme solution of the myrosinase. This treatment removes an enzyme activity of epithiospecifier protein (ESP) while retaining most of the myrosinase activity.

### <Preparation of the Pharmaceutical Composition in the Present Disclosure>

The glucoraphanin extract and the myrosinase extract are mixed to prepare the pharmaceutical composition of the present disclosure. Preferably, the glucoraphanin extract and the myrosinase extract are made into powders and then mixed in a powder form to prepare the composition, thereby making it easy to store and maintain the activity of glucoraphanin, and maintain the stability of the enzyme activity of myrosinase. The composition in powder form is easy to be formulated into a tablet, a capsule, a pulvis, a powder, a water-soluble granule, and a granule, and is also easy to be mixed with other additives to prepare an ointment, a patch, an emulsion, a liniment, a paste, an injection, a spray, a cream, a lotion, an oil, a suspension, a gel, or a tonic.

The powdered glucoraphanin extract and myrosinase extract can be prepared using conventional methods in the art: from the perspective of maintaining the activity of glucoraphanin and maintaining the stability of the enzyme activity of myrosinase, a freeze-drying method is preferably used.

During the freeze-drying, the glucoraphanin extract and the myrosinase extract can be mixed with an additive and water separately, and then freeze-dried to obtain a mixture of a powdered glucoraphanin extract and the additive as well as a mixture of a powdered myrosinase extract and the additive.

The additive includes any pharmaceutically acceptable carrier and/or excipient known in the art, as long as it does not affect the effects of the present disclosure achieved by the glucoraphanin and myrosinase. For example, lactose, glucose, sucrose, sorbitol, mannitol, starch, dextrin, acacia, calcium phosphate, alginate, gelatin, calcium silicate, microcrystalline cellulose, polyvinylpyrrolidone, water, syrup, methylcellulose, methylparaben, propylparaben, talc, magnesium stearate, super sodium starch glycolate, vegetable oil, animal oil, and mineral oil can be used.

In the present disclosure, in addition to the above ingredients, the pharmaceutical composition may also include other additives, such as lubricants, wetting agents, sweeteners, flavors, emulsifiers, suspending agents, and preservatives. As long as it does not affect the effect of the present disclosure achieved by the glucoraphanin and myrosinase, any additive can be added.

The glucoraphanin content in the composition or extract can be determined by conventional methods in the art, for example, high-performance liquid chromatography (HPLC) and the like. The enzyme activity of the myrosinase can also be determined by conventional methods in the art, for example, measuring an initial velocity of the enzymatic reaction.

### <Use of the Pharmaceutical Composition in the Present Disclosure>

In the present disclosure, the pharmaceutical composition has a wide range of applications and can be applied to many fields, such as health care, food, or hair care. As long as the pharmaceutical composition can be used to improve and treat leukotrichia and/or alopecia, its application scope is not limited thereto.

In the present disclosure, the pharmaceutical composition has a variety of administration forms, such as oral administration, injection, skin administration, or mucosal administration, as long as it can achieve the effect of improving and treating leukotrichia and/or alopecia.

In the present disclosure, the dosage, administration frequency, and administration time of the pharmaceutical composition may vary according to the subject's leukotrichia and/or alopecia symptoms, the subject's age, and the pharmaceutical dosage form. Preferably, the pharmaceutical composition is orally administered based on the amount of glucoraphanin at (5-500) mg/person/day, more preferably (5-100) mg/person/day, and most preferably (10-60) mg/person/day.

The present disclosure is further described below with reference to specific examples, but the protection scope the present disclosure is not limited thereto.

### EXAMPLE

### I. Preparation of a Glucoraphanin Extract

### A. Crushing of Raw Materials

10 kg of radish seeds were crushed using a crusher and a resulting seed powder was collected.

### B. Extraction of Glucoraphanin

100 L of deionized water was heated to boiling, and the raw materials crushed in step A were added to the deionized water at a solid-to-liquid ratio of 1:10. A resulting mixture was extracted with boiling water for 10 min, and then filtered with filter cloth to obtain a water extract: the extraction was repeated once and 200 L of a glucoraphanin water extract was collected.

10 kg of activated carbon was added to the glucoraphanin water extract, stirred and adsorbed at 25° C. for 30 min, and filtered to obtain an activated carbon filter cake with glucoraphanin adsorbed. 100 L of a 10% ethanol aqueous solution was added to the activated carbon filter cake with glucoraphanin adsorbed to obtain a mixed solution. The mixed solution was added with ammonia water and determined by a pH meter to have a pH value of 10, eluted by stirring at 25° C. for 30 min, and filtered to obtain activated carbon and an eluate: the eluate was distilled under reduced pressure and freeze-dried to obtain 200 g of the glucoraphanin extract, where a purity of glucoraphanin was measured to be 95%.

### II. Preparation of a Myrosinase Extract

1 kg of seeds of cruciferous plants not treated by high temperature are added into 10 L of deionized water, ultrasonically broken, and filtered with gauze to obtain a clear liquid. The myrosinase in the clear liquid was precipitated using a (NH₄)₂SO₄ aqueous solution with a saturation of 55%. An obtained myrosinase mixture containing precipitate was centrifuged at 10,000 r/min for 20 min, and a supernatant was discarded to obtain 300 g of a moist precipitate containing myrosinase: the precipitate was dialyzed overnight using a dialysis membrane (specifications: 8,000 KD to 14,000 KD) to obtain a liquid myrosinase extract in the dialysis membrane, which was 3,000 g of an enzyme liquid. The enzyme liquid was subjected to enzyme activity measurement, and a measured unit enzyme activity was 40 mU/g, and a total enzyme activity was 120 U.

### III. Preparation of a Composition Containing Glucoraphanin and Myrosinase as Active Ingredients

The glucoraphanin and myrosinase extracts prepared by the above method were used as raw materials, mixed with other additives in the following specific examples to prepare the composition of the present disclosure in the form of water-soluble granules, tablets, and capsules.

### IV. Determination of a Glucoraphanin Content

The glucoraphanin content was determined by HPLC, and the data was calculated using a standard curve method. The detection conditions of HPLC were as follows:

Mobile phase:
A: methanol (chromatography grade), B: purified water (containing 0.02% TFA).
Stationary phase:
Diamonsil C-18 analytical column from Dikma.
Detection conditions:
Isocratic elution, A: B=5%: 95%, flow rate 1 mL/min, column temperature 30° C., detection wavelength 235 nm.

### V. Determination of Enzyme Activity of Myrosinase

100 mL of a reaction system was prepared, including PBS with pH=6.5 and glucoraphanin, where the glucoraphanin had a concentration of 1 mg/mL. The reaction system was placed in a 200 ml beaker at 37° C., a sample was taken and a time was recorded as time 0.1 mg of the myrosinase extract was added to the reaction system, and samples were taken every 1 min. HCl was added to each sample to stop the reaction, and the glucoraphanin content of the sample was detected by HPLC, where a detection method referred to IV. Determination of a glucoraphanin content. A graph was plotted with time as an abscissa and absorbance as an ordinate to calculate the enzyme activity.

The enzyme activity of myrosinase was defined as: an amount of enzyme required to catalyze 1 μmol of glucoraphanin per minute at pH=6.5 and 37° C., in mU/g.

The total enzyme activity of myrosinase was defined as: an enzyme activity obtained by multiplying the myrosinase extract (g) contained in the composition with the enzyme activity per gram of myrosinase extract (mU/g), in mU.

### Example 1 Preparation of Water-Soluble Granule 1 Containing Glucoraphanin and Myrosinase

200 g of the glucoraphanin extract prepared by the method in I (the glucoraphanin had a purity of 95%) and 19.8 kg of dextrin were mixed and dissolved in 100 L of deionized water, and then spray-dried. The spray drying was conducted at an air inlet temperature of 180° C., an air outlet temperature of 80° C., and a liquid inlet speed of 5 L/h, and a product was collected to prepare a spray-dried powder of the glucoraphanin. 200 g of the myrosinase extract prepared by the method in II and 19.8 kg of dextrin were mixed and dissolved in 100 L of deionized water, and then spray-dried. The spray drying was conducted at an air inlet temperature of 180° C., an air outlet temperature of 80° C., and a liquid inlet speed of 5 L/h, and a product was collected to prepare a spray-dried powder of the myrosinase. The spray-dried powder of the glucoraphanin and the spray-dried powder of the myrosinase were mixed to obtain a composition containing 0.5% of the glucoraphanin extract and 0.5% of the myrosinase extract (the myrosinase had a unit enzyme activity of 33 mU/g). The composition was granulated using a granulator, and the water-soluble granule 1 was obtained by packaging with an automatic granule packaging machine in an amount containing 10 mg of the glucoraphanin per package, where the myrosinase was determined to have a total enzyme activity of 0.35 mU.

### Example 2 Preparation of Water-Soluble Granule 2 Containing Glucoraphanin and Myrosinase

300 g of the glucoraphanin extract prepared by the method in I (the glucoraphanin had a purity of 92%) and 4.7 kg of dextrin were mixed and dissolved in 2.5 L of deionized water, and then spray-dried. The spray drying was conducted at an air inlet temperature of 180° C., an air outlet temperature of 80° C., and a liquid inlet speed of 5 L/h, and a product was collected to prepare a spray-dried powder of the glucoraphanin. 300 mg of the myrosinase extract prepared by the method in II and 4.7 kg of dextrin were mixed and dissolved 2.5 L of deionized water, and then spray-dried. The spray drying was conducted at an air inlet temperature of 180° C., an air outlet temperature of 80° C., and a liquid inlet speed of 5 L/h, and a product was collected to prepare a spray-dried powder of the myrosinase. The spray-dried powder of the glucoraphanin and the spray-dried powder of the myrosinase were mixed to obtain a composition containing 3% of the glucoraphanin extract and 3% of the myrosinase extract (the myrosinase had a unit enzyme activity 30 mU/g). The composition was granulated using a granulator, and the water-soluble granule 2 was obtained by packaging with an automatic granule packaging machine in an amount containing 30 mg of the glucoraphanin per package, where the myrosinase was determined to have a total enzyme activity of 0.98 mU.

### Example 3 Preparation of Water-Soluble Granule 3 Containing Glucoraphanin and Myrosinase

100 g of the glucoraphanin extract prepared by the method in I (the glucoraphanin had a purity of 90%) and 4.9 kg of dextrin were mixed and dissolved 500 L of deionized water, and then spray-dried. The spray drying was conducted at an air inlet temperature of 180° C. an air outlet temperature of 80° C. and a liquid inlet speed of 5 L/h, and a product was collected to prepare a spray-dried powder of the glucoraphanin. 100 mg of the myrosinase extract prepared by the method in II and 4.9 kg of dextrin were mixed and dissolved in 100 L of deionized water, and then spray-dried. The spray drying was conducted at an air inlet temperature of 180° C. an air outlet temperature of 80° C. and a liquid inlet speed of 5 L/h, and a product was collected to prepare a spray-dried powder of the myrosinase. The spray-dried powder of the glucoraphanin and the spray-dried powder of the myrosinase were mixed to obtain a composition containing 1% of the glucoraphanin extract and 1% of the myrosinase extract (the myrosinase had a unit enzyme activity of 31 mU/g). The composition was granulated using a granulator, and the water-soluble granule 3 was obtained by packaging with an automatic granule packaging machine in an amount containing 10 mg of the glucoraphanin per package, where the myrosinase was determined to have a total enzyme activity of 0.34 mU.

### Example 4 Preparation of Tablet 1 Containing Glucoraphanin and Myrosinase

300 g of the glucoraphanin extract prepared by the method in I (the glucoraphanin had a purity of 91%) and 1.7 kg of dextrin were mixed and dissolved in 10 L of deionized water, and then spray-dried in a same manner as that in Example 1 to prepare a spray-dried powder of glucoraphanin with a 15% glucoraphanin content. 300 g of the myrosinase extract prepared by the method in II and 1.7 kg of dextrin were mixed and dissolved in 10 L of deionized water, and then spray-dried in a same manner as that in Example 1 to prepare a spray-dried powder of myrosinase with a 15% myrosinase content. The spray-dried powder of the glucoraphanin and the spray-dried powder of the myrosinase were mixed. 100 g of magnesium stearate. 1.000 g of Peng Su Wang. and 4.9 kg of microcrystalline cellulose were added, and mixed evenly to obtain a composition containing 3% of the glucoraphanin extract and 3% of the myrosinase extract (the myrosinase had a unit enzyme activity of 35 mU/g). The composition was dry-granulated, and then granulated drug-containing granules were tableted into the tablet 1 each containing 15 mg of glucoraphanin, where the myrosinase had a total enzyme activity of 0.58 mU.

### Example 5 Preparation of Tablet 2 Containing Glucoraphanin and Myrosinase

600 g of the glucoraphanin extract prepared by the method in I (the glucoraphanin had a purity of 91%) and 1.4 kg of dextrin were mixed and dissolved in 10 L of deionized water, and then spray-dried in a same manner as that in Example 1 to prepare a spray-dried powder of glucoraphanin with a 30% glucoraphanin content. 600 g of the myrosinase extract prepared by the method in II and 1.4 kg of dextrin were mixed and dissolved in 10 L of deionized water, and then spray-dried in a same manner as that in Example 1 to prepare a spray-dried powder of myrosinase with a 30% myrosinase content. The spray-dried powder of the glucoraphanin and the spray-dried powder of the myrosinase were mixed. 100 g of magnesium stearate. 1.000 g of Peng Su Wang. and 4.9 kg of microcrystalline cellulose were added, and mixed evenly to obtain a composition containing 6% of the glucoraphanin extract and 6% of the myrosinase extract (the myrosinase had a unit enzyme activity of 33 mU/g). The composition was dry-granulated, and then granulated drug-containing granules were tableted into the tablet 2 each containing 30 mg of glucoraphanin, where the myrosinase had a total enzyme activity of 1.09 mU.

### Example 6 Preparation of Tablet 3 Containing Glucoraphanin and Myrosinase

400 g of the glucoraphanin extract prepared by the method in I (the glucoraphanin had a purity of 92%) and 1.6 kg of dextrin were mixed and dissolved in 5 L of deionized water, and then spray-dried in a same manner as that in Example 1 to prepare a spray-dried powder of glucoraphanin with a 20% glucoraphanin content. 400 g of the myrosinase extract prepared by the method in II and 1.6 kg of dextrin were mixed and dissolved in 10 L of deionized water, and then spray-dried in a same manner as that in Example 1 to prepare a spray-dried powder of myrosinase with a 20% myrosinase content. The spray-dried powder of the glucoraphanin and the spray-dried powder of the myrosinase were mixed. 100 g of magnesium stearate. 1 kg of Peng Su Wang. and 4.9 kg of microcrystalline cellulose were added, and mixed evenly to obtain a composition containing 4% of the glucoraphanin extract and 4% of the myrosinase extract (the myrosinase had a unit enzyme activity of 31 mU/g). The composition was dry-granulated, and then granulated drug-containing granules were tableted into the tablet 3 each containing 20 mg of glucoraphanin, where the myrosinase had a total enzyme activity of 0.67 mU.

### Example 7 Preparation of Tablet 4 Containing Glucoraphanin and Myrosinase

A glucoraphanin extract was prepared by the method in I, such that the glucoraphanin had a purity of 94%. A myrosinase extract was prepared by the method in II, such that the myrosinase had a unit enzyme activity of 38 mU/g.

The tablet 4 containing glucoraphanin and myrosinase was prepared in a same manner as that in Example 4, and added amounts of the glucoraphanin extract and myrosinase extract were adjusted such that each tablet 4 included 10 mg of glucoraphanin, and the myrosinase had a total enzyme activity of 0.40 mU.

### Comparative Example 1 Preparation of Water-Soluble Granule 4 Containing Glucoraphanin and Myrosinase

The water-soluble granule containing glucoraphanin and myrosinase was prepared in a same manner as that in Example 1, and added amounts of the glucoraphanin extract and myrosinase extract were adjusted (the glucoraphanin had a purity of 91%), and the water-soluble granule 4 containing 0.04% of glucoraphanin extract and 0.01% of myrosinase extract was prepared (the myrosinase had a unit enzyme activity of 32 mU/g), such that each water-soluble granule included 10 mg of glucoraphanin, and the myrosinase had a total enzyme activity of 0.088 mU.

### Comparative Example 2 Preparation of Tablet 5 Containing Glucoraphanin and Myrosinase

The tablet containing glucoraphanin and myrosinase was prepared in a same manner as that in Example 4, and added amounts of the glucoraphanin extract and myrosinase extract were adjusted (the glucoraphanin had a purity of 92%), and the tablet 5 containing 0.2% of glucoraphanin extract and 0.02% of myrosinase extract was prepared (the myrosinase had a unit enzyme activity of 35 mU/g), such that each tablet included 15 mg of glucoraphanin, and the myrosinase had a total enzyme activity of 0.057 mU.

### Comparative Example 3 Preparation of Tablet 6 Containing Glucoraphanin and Myrosinase

The tablet containing glucoraphanin and myrosinase was prepared in a same manner as that in Example 5, and added amounts of the glucoraphanin extract and myrosinase extract were adjusted (the glucoraphanin had a purity of 90%), and the tablet 6 containing 0.05% of glucoraphanin extract and 1% of myrosinase extract was prepared (the myrosinase had a unit enzyme activity of 31 mU/g), such that each tablet included 0.5 mg of glucoraphanin, and the myrosinase had a total enzyme activity of 0.34 mU.

<Improvement and Treatment of Leukotrichia and/or Alopecia by the Composition of the Present Disclosure>

The following experiments were conducted using the water-soluble granules and tablets of the composition containing glucoraphanin and myrosinase prepared in Examples 1 to 7 and Comparative Examples 1 to 3 to investigate the improvement and therapeutic effect of the composition on leukotrichia and/or alopecia.

In the experiments, through voluntary participation, voluntary subjects who met the following conditions after physical examination were selected: (1) people with significant hair whitening and leukotrichia lasting for more than 3 months: (2) there were no obvious symptoms of discomfort related to leukotrichia, and an influence of other diseases or factors on leukotrichia and/or alopecia had been ruled out: (3) aging from 35 to 78 years old: (4) with ability to answer or understand the self-photography guide for leukotrichia conditions: (5) those who had obtained informed consent and could cooperate with follow-up visits.

Meanwhile, the following exclusion criteria were set: (1) subjects who had organic (caused by certain organs of the body) and external environmental interference factors that affect leukotrichia and/or alopecia: (2) subjects with severe primary diseases or severe mental illness that affected leukotrichia and/or alopecia: (3) those who had allergies or were allergic to the test substances, and those who took drugs that were related to the tested functions in a short period of time and could affect the determination results: (4) those who failed to use the test samples in accordance with regulations and could not determine the efficacy, or had incomplete data that affected the determination of efficacy or safety: (5) those under 35 years old or over 78 years old, and women who were pregnant or breastfeeding.

This trial adopted a before-and-after control design to collect picture data and information on the degree of leukotrichia from the subjects before and after taking drugs. In addition, various factors that affected the results were also taken into account: gender, age, sleeping habits, and occupational characteristics, to determine a correlation between the improvement in leukotrichia and/or alopecia and the product intake. During the test, subjects were required to take the water-soluble granules or tablets of the composition containing glucoraphanin and myrosinase prepared in the present disclosure after meals. The glucoraphanin was orally administered at (10-100) mg/person/day for 1 to 3 months, where a frequency of administration could be appropriately adjusted according to the specific conditions of the subjects, and was generally 1 to 3 times/day. The dosage was determined by the following factors: the subject's weight, age, severity of leukotrichia and/or alopecia, and acceptance of the drug.

Table 1a, Table 1b, Table 1c, FIG. 1, and FIG. 2 showed the improvement and treatment effects of leukotrichia and/or alopecia after subjects took the compositions containing glucoraphanin and myrosinase prepared in Examples 1 to 7 and Comparative Examples 1 to 3. Table 1a showed studies conducted on male subjects: Table 1b showed studies conducted on female subjects: Table 1c investigated effects of the content of glucoraphanin and the total enzyme activity of myrosinase on the improvement and therapeutic effect of leukotrichia and/or alopecia.

Table 2 showed the improvement and therapeutic effects of leukotrichia and/or alopecia from the perspective of hair growth effect and hair blackening effect after a large range of 191 subjects who met the above conditions took tablet 4 of the composition in Example 7 of the present disclosure. The dosage, frequency, and time of administration were based on the aforementioned standards.

It should be noted that in the above tables, “improvement start time” meant the time when white hair turned black and/or alopecia reduction effect began to appear, and women mainly show whether alopecia occurred when combing hair. “Whether other drugs were used” meant whether the subject had ever taken other drugs to improve or treat leukotrichia and/or alopecia. “Obvious” meant that hair growth and hair blackening increased significantly, and the area of hair growth or hair blackening was greater than 25% of the total hair area; “Normal” meant that hair growth and hair blackening had increased, and the area of hair growth or hair blackening was 1% to 25% of the total hair area; “Ineffective” meant that there was no obvious effect, and the area of hair growth or hair blackening was less than 1% of the total hair area.

TABLE 1a

| No. | 1 | 2 | 3 | 4 | 5 |
|---|---|---|---|---|---|
| Gender | Male | Male | Male | Male | Male |
| Age | 35 | 36 | 52 | 45 | 78 |
| Incidence | 30 | 25 | 38 | 35 | 60 |
| age | | | | | |
| Alopecia | None | None | None | Head top | Forehead |
| range | | | | | |
| Baldness | None | None | None | None | Yes |
| family | | | | | |
| history | | | | | |
| White hair | Head, about ⅓ | Head, about 1/10 | Head, about ½ | Head, about ½ | Head, about ½ |
| range | of white hair | of white hair | of white hair | of white hair | of white hair |
| White hair | None | Yes | None | None | Yes |
| family | | | | | |
| history | | | | | |
| Dosage and | 10 mg/d, | 30 mg/d, | 30 mg/d, | 30 mg/d, | 20 mg/d, |
| dosage form | water-soluble | water-soluble | tablet 1 | tablet 2 | tablet 3 |
| | granule 1 | granule 2 | | | |
| Improvement | 20 d | 1 month | 15 d | 15 d | 1 month |
| start time | | | | | |
| Improvement | While hair | While hair | While hair | While hair blackening | While hair |
| effect | blackening appeared | blackening appeared | blackening appeared | appeared and alopecia | blackening appeared |
| | after 20 d; about ⅓ | after 1 month; about | after 15 d; about ½ | reduced after 15 d; about | and alopecia |
| | white hair turned | ⅓ white hair | white hair turned | ½ white hair turned | reduced after 1 |
| | black after 1 month; | turned black after 2 | black after 1 month; | black after 1 month; about | month; about ⅓ |
| | about ½ white hair | months; about ⅔ | about ⅔ white hair | ⅔ white hair turned | white hair turned |
| | turned black after 2 | white hair turned | turned black after 2 | black and alopecia | black and alopecia |
| | months; about ⅔ | black after 3 | months; about ¾ | significantly reduced after | significantly |
| | white hair turned | months; | white hair turned | 2 months; almost all white | reduced after 2 |
| | black after 3 | | black after 3 | hair turned black, with | months; about ½ |
| | months; | | months; | basically no alopecia after | white hair turned |
| | | | | 3 months | black and alopecia |
| | | | | | reduced after 3 |
| | | | | | months |
| Whether | None | None | None | None | No use of |
| other drugs | | | | | other drugs |
| were used | | | | | within 5 years |

TABLE 1b

| No. | 1 | 2 | 3 | 4 | 5 |
|---|---|---|---|---|---|
| Gender | Female | Female | Female | Female | Female |
| Age | 35 | 38 | 44 | 57 | 60 |
| Incidence | 32 | 25 | 40 | 45 | 55 |
| age | | | | | |
| Alopecia | Head, more | None | Head top, | Head, | Head, |
| range | alopecia | | sparse hair | more | more |
| | | | and prone to | alopecia | alopecia |
| | | | alopecia | | |
| Family | None | None | Yes | None | None |
| history of | | | | | |
| baldness | | | | | |
| White hair | None | Head, about ⅕ | None | Head, about ⅓ | Head, about ½ |
| range | | of white hair | | of white hair | of white hair |
| Family | None | Yes | None | None | Yes |
| history of | | | | | |
| leukotrichia | | | | | |
| Dosage and | 30 mg/d, | 30 mg/d, | 15 mg/d, | 30 mg/d, | 20 mg/d, |
| dosage form | water-soluble | water-soluble | tablet 1 | tablet 2 | tablet 3 |
| | granule 1 | granule 3 | | | |
| Improvement | 5 d | 15 d | 15 d | 15 d | 1 month |
| start time | | | | | |
| Improvement | Slightly reduced | While hair | Reduced alopecia | Reduced alopecia after | Reduced alopecia and while |
| effect | alopecia after 5 | blackening | after 15 d; | 15 d; white hair | hair blackening appeared |
| | d; significantly | appeared after 15 | significantly reduced | blackening appeared; | after 1 month; about ⅓ |
| | reduced alopecia | d; about ¼ while | alopecia after 1 | about ⅓ white hair | white hair turned black and |
| | after 10 d; | hair turned black | month; basically no | turned black and | alopecia significantly |
| | basically no | after 1 month; | alopecia after 2 | alopecia significantly | reduced after 2 months; |
| | alopecia after 1 | about ½ white | months. | reduced after 2 months; | about ½ white hair turned |
| | month | hair turned black | | about ½ white hair | black and alopecia reduced |
| | | after 3 months | | turned black and | after 3 months |
| | | | | alopecia reduced after 3 | |
| | | | | months | |
| Whether | None | None | None | Using traditional | None |
| other drugs | | | | Chinese medicine for | |
| were used | | | | hair growth 5 years ago, | |
| | | | | and there was no little | |
| | | | | effect for two years | |

TABLE 1c

| No. | 1 | | 2 | | 3 | |
|---|---|---|---|---|---|---|
| Gender | Female | | Male | | Female | |
| Age | 40 | | 35 | | 55 | |
| Incidence | 35 | | 32 | | 45 | |
| age | | | | | | |
| Alopecia | Head, more | | None | | None | |
| range | alopecia | | | | | |
| Family | None | | None | | None | |
| history of | | | | | | |
| baldness | | | | | | |
| White hair | None | | Head, about 1/10 | | Head, about ⅕ | |
| range | | | of white hair | | of white hair | |
| Family | None | | Yes | | None | |
| history of | | | | | | |
| leukotrichia | | | | | | |
| Dosage and | 20 mg/d, | 20 mg/d, | 30 mg/d, | 30 mg/d, | 2 mg/d, | 30 mg/d, |
| dosage form | water-soluble | water-soluble | tablet 5 | tablet 1 | tablet 6 | tablet 2 |
| | granule 4 | granule 1 | | | | |
| Improvement | 3 months | 10 d | None | 15 d | 2 months | 15 d |
| start time | | | | | | |
| Improvement | Slightly | Reduced alopecia after | Slightly | While hair blackening | While hair | While hair |
| effect | reduced | 10 d; about ⅓ white | reduced | appeared after 15 d; | blackening | blackening |
| | alopecia | hair turned black after | alopecia | about ⅓ white hair | appeared after | appeared after 15 |
| | after 2 | 1 month; about ⅔ | after 3 | turned black after 1 | 2 months; | d; about ½ white |
| | months | white hair turned black | months | month; about ½ white | about 1/10 | hair turned black |
| | | after 2 months; | | hair turned black after 2 | white hair | after 2 months; |
| | | basically no alopecia | | months; about ¾ white | turned black | about ⅔ white |
| | | after 3 months | | hair turned black after 3 | after 3 months | hair turned black |
| | | | | months; | | after 3 months; |
| Whether | None | | None | | None | |
| other drugs | | | | | | |
| were used | | | | | | |

TABLE 2

| Use | Number of | Hair growth effect | | | Hair blackening effect | | |
|---|---|---|---|---|---|---|---|
| time | subjects | Obvious | Normal | Ineffective | Obvious | Normal | Ineffective |
| 3 months | 99 | 63 | 27 | 9 | 64 | 29 | 6 |
| | (male) | 63.64% | 27.27% | 9.09% | 64.65% | 29.29% | 6.06% |
| | 92 | 59 | 22 | 11 | 75 | 5 | 12 |
| | (female) | 64.13% | 23.91% | 11.96% | 81.52% | 5.43% | 13.05% |

Table 1a and Table 1b showed that for subjects of different ages and genders, suffering from different degrees of leukotrichia and/or alopecia, after oral administration of glucoraphanin at a dose of (10-30) mg/person/day, there were varying degrees of improvement between 5 d and 3 months. For patients with leukotrichia, such as the subject No. 3 in Table 1a: after taking the tablet 1 at 30 mg/day for 15 d, some of white hair began to turn black; and about ½ of the white hair turned black after 1 month. This indicated a significant effect, which was seen in the before-and-after comparison of A and B in FIG. 1. In addition, the subject suffering from both leukotrichia and alopecia shown in No. 4 in Table 1b, after oral administration of tablet 2 at 30 mg/day: reduced alopecia and white hair blackening appeared on day 15: after 2 months, about ⅓ of white hair turned black, and alopecia was significantly reduced: after 3 months, about ½ of white hair turned black, and there was less alopecia. This subject had been treated with other traditional Chinese medicines 5 years ago, but the leukotrichia and alopecia did not improve after two years of continuous treatment.

a and b in FIG. 2 also showed that the subject with leukotrichia and alopecia shown in No. 4 of Table 1a, took Tablet 2 at 30 mg/day: after 15 d, the white hair began to turn black and the alopecia decreased: after 1 month, about ½ of white hair turned black: after 3 months, almost all white hair turned black, and there was basically no alopecia. This indicated that the combination of glucoraphanin and myrosinase was extremely effective in treating leukotrichia and alopecia.

In addition, Table 1c also showed that when the subject of No. 1 took the water-soluble granule 4, the subject of No. 2 took the tablet 5, and the subject of No. 3 took the tablet 6: the myrosinase in the water-soluble granule 4 and tablet 5 had an extremely low total enzyme activity, being 0.088 mU and 0.057 mU, respectively, and were not within the range of 0.1 mU to 10 U of the present disclosure. Therefore, the granule and tablet could not effectively catalyze the enzymatic reaction of glucoraphanin, resulting in poor therapeutic effect on leukotrichia and/or alopecia. Similarly, for the tablet 6, the content of glucoraphanin was 0.05%, which was not within the range of 0.1% to 50% of the present disclosure: a daily dose was 2 mg/day, which was also not in the range of (5-500) mg/day. Therefore, too low substrate content resulted in less products obtained after the enzymatic reaction, which could not effectively improve and treat leukotrichia. However, when the subject of No. 1 was given the water-soluble granule 1, the subject of No. 2 was given the tablet 1, and the subject of No. 3 was given the tablet 2: the content of glucoraphanin and the total enzyme activity of myrosinase in these compositions were both within the scope of the present disclosure, and a daily dosage was also within the range of (5-500) mg/day. As a result, significant improvement and treatment effects occurred on leukotrichia and/or alopecia.

During the experiments, it was found that a difference in dosage form showed no significant impact on the improvement and treatment effects of leukotrichia and/or alopecia.

Table 2 showed that after the subjects took the composition containing glucoraphanin and myrosinase for 3 months, more than 60% of the subjects had obvious hair growth and hair blackening effects. This proved that the pharmaceutical composition of the present disclosure could effectively improve and treat leukotrichia and/or alopecia.

### INDUSTRIAL APPLICABILITY

In the present disclosure, the pharmaceutical composition including glucoraphanin and myrosinase as active ingredients is safe, rapid, and effective in improving and treating leukotrichia and/or alopecia, and can be applied to subjects of different ages and symptoms. After subjects take the pharmaceutical composition, the improvement effect of leukotrichia and/or alopecia begins to appear in a short period of 5 d to 15 d; and after 3 months, most of the white hair turns black while the alopecia is also improved to a greater extent. Therefore, the pharmaceutical composition of the present disclosure has desirable application prospects in improving and treating the leukotrichia and/or alopecia.

1. A pharmaceutical composition for improving and treating leukotrichia and/or alopecia, comprising glucoraphanin and myrosinase as active ingredients, wherein the glucoraphanin has a content of 0.1% to 50% and the myrosinase has a total enzyme activity of 0.1 mU to 10 U based on a total weight of the pharmaceutical composition.

2. The pharmaceutical composition according to claim 1, wherein the glucoraphanin has a content of preferably 0.5% to 10%, more preferably 1% to 5%.

3. The pharmaceutical composition according to claim 1, wherein the myrosinase has a total enzyme activity of preferably 0.3 mU to 5 U, more preferably 0.5 mU to 1 U.

4. The pharmaceutical composition according to claim 1, wherein the pharmaceutical composition is subjected to oral administration at a dosage of 5 mg/person/day to 500 mg/person/day based on an amount of the glucoraphanin.

5-13. (canceled)

14. The pharmaceutical composition according to claim 2, wherein the pharmaceutical composition is subjected to oral administration at a dosage of 5 mg/person/day to 500 mg/person/day based on an amount of the glucoraphanin.

15. The pharmaceutical composition according to claim 3, wherein the pharmaceutical composition is subjected to oral administration at a dosage of 5 mg/person/day to 500 mg/person/day based on an amount of the glucoraphanin.

16. The pharmaceutical composition according to claim 4, wherein the pharmaceutical composition is subjected to oral administration at a dosage of preferably 5 mg/person/day to 100 mg/person/day, more preferably 10 mg/person/day to 60 mg/person/day based on the amount of the glucoraphanin.

17. The pharmaceutical composition according to claim 14, wherein the pharmaceutical composition is subjected to oral administration at a dosage of preferably 5 mg/person/day to 100 mg/person/day, more preferably 10 mg/person/day to 60 mg/person/day based on the amount of the glucoraphanin.

18. The pharmaceutical composition according to claim 15, wherein the pharmaceutical composition is subjected to oral administration at a dosage of preferably 5 mg/person/day to 100 mg/person/day, more preferably 10 mg/person/day to 60 mg/person/day based on the amount of the glucoraphanin.

19. The pharmaceutical composition according to claim 1, wherein the glucoraphanin and the myrosinase each are derived from one or more selected from the group consisting of a natural product extract, a microbial extract, a biological synthesis product, and a chemical synthesis product.

20. The pharmaceutical composition according to claim 2, wherein the glucoraphanin and the myrosinase each are derived from one or more selected from the group consisting of a natural product extract, a microbial extract, a biological synthesis product, and a chemical synthesis product.

21. The pharmaceutical composition according to claim 3, wherein the glucoraphanin and the myrosinase each are derived from one or more selected from the group consisting of a natural product extract, a microbial extract, a biological synthesis product, and a chemical synthesis product.

22. The pharmaceutical composition according to claim 4, wherein the glucoraphanin and the myrosinase each are derived from one or more selected from the group consisting of a natural product extract, a microbial extract, a biological synthesis product, and a chemical synthesis product.

23. The pharmaceutical composition according to claim 19, wherein the natural product extract is extracted from a natural product being one or more selected from the group consisting of Brassica oleracea var. gemmifera Zenker, Brassica oleracea var. capitata Linnaeus, Brassica oleracea var. botrytis Linnaeus, Brassica rapa var. glabra Regel, kale, Brassica oleracea var. acephala DC., broccoli sprouts, Brassica oleracea var. albiflora Kuntze, cauliflower, Brassica juncea var. napiformis Pailleux et Bois, mustard, Brassica rapa L., Raphanus sativus L., Eruca vesicaria subsp. sativa (Miller) Thellung, and Nasturtium officinale R. Br. ex W. T. Aiton.

24. The pharmaceutical composition according to claim 1, wherein the pharmaceutical composition is in the form of a tablet, a capsule, a pulvis, a powder, a water-soluble granule, a granule, an ointment, a patch, an emulsion, a liniment, a paste, an injection, a spray, a cream, a lotion, an oil, a suspension, a gel, or a tonic.

25. The pharmaceutical composition according to claim 1, wherein the pharmaceutical composition is administered to a subject by oral administration, injection, skin administration, or mucosal administration.

26. A preparation method of a pharmaceutical composition for improving and treating leukotrichia and/or alopecia, comprising: encapsulating glucoraphanin and myrosinase as active ingredients into the pharmaceutical composition, wherein the glucoraphanin has a content of 0.1% to 50% and the myrosinase has a total enzyme activity of 0.1 mU to 10 U based on a total weight of the pharmaceutical composition.

27. The preparation method of a pharmaceutical composition according to claim 26, comprising the following steps:
preparing a glucoraphanin extract;
preparing a myrosinase extract;
mixing the glucoraphanin extract, an additive, and water to allow spray-drying to prepare a dried glucoraphanin powder;
mixing the myrosinase extract, the additive, and water to allow spray-drying to prepare a dried myrosinase powder; and
mixing the dried glucoraphanin powder and the dried myrosinase powder to obtain the pharmaceutical composition.

28. The preparation method of a pharmaceutical composition according to claim 26, wherein the pharmaceutical composition is subjected to granulation by a granulator and then sub-packaging or tableting.

29. A method for improving and treating leukotrichia and/or alopecia using glucoraphanin and myrosinase.
